(19) United States
(12) Patent Application Publication
(10) Pub. No.: US 2014/0066881 A1
(43) Pub. Date: Mar. 6, 2014

(54) CARDIAC RESUSCITATION METHODS AND KITS

(71) Applicant: ZOLL MEDICAL CORPORATION, Chelmsford, MA (US)

(72) Inventor: Gary A. Freeman, Newton Center, MA (US)

(73) Assignee: ZOLL MEDICAL CORPORATION, Chelmsford, MA (US)

(21) Appl. No.: 13/798,348

(22) Filed: Mar. 13, 2013

Related U.S. Application Data
(60) Provisional application No. 61/695,876, filed on Aug. 31, 2012.

(51) Int. Cl. A61K 9/00; A61N 1/39

(52) U.S. Cl. CPC A61K 9/0073; A61N 1/39
USPC 604/500; 514/12.4

(57) ABSTRACT

The invention provides methods of improving circulation in a subject undergoing a cardiac resuscitation protocol, reducing the risk of stroke in a subject undergoing a cardiac resuscitation protocol, improving the efficacy of cardiac resuscitation in a subject undergoing a cardiac resuscitation protocol, and providing cardiac resuscitation to a subject having cardiac arrest. Also provided are kits including a composition containing at least one vasoconstrictor formulated for systemic administration, and a composition containing at least one vasodilator formulated for nasal administration.

### CLAIM OF PRIORITY

This application claims priority under 35 USC §119(e) to U.S. Patent Application Ser. No. 61/695,876, filed on Aug. 31, 2012, the entire contents of which are hereby incorporated by reference.

### TECHNICAL FIELD

This invention relates to methods of performing cardiac resuscitation and kits for use in the performance of cardiac resuscitation.

### BACKGROUND

Sudden cardiac arrest (colloquially “heart attack”) is a regular killer. The best treatment for cardiac arrest is quick and competent chest compressions to keep blood flowing through a victim's heart. Generally, every minute of delay in treating a cardiac arrest victim lowers the chance of survival of the victim by about ten percent. As a result, the ability to provide cardiac resuscitation in a competent manner can be a very important personal skill, and is particularly important for professional healthcare workers, such as emergency medical technicians (EMTs).

The current standard for the treatment of a patient in cardiac arrest includes the administration of a pharmaceutical agent that increases blood volume, increases blood pressure, or prevents arrhythmia in the patient.

### SUMMARY

Current pharmacological treatments of a subject in cardiac arrest during a resuscitation event include the administration of a pharmaceutical agent that increases blood volume, increases blood pressure (e.g., a vasoconstrictor), or decreases the risk of arrhythmia in the subject. These current treatments focus on the administration of agents that constrict blood vessels, and result in an increase in the blood pressure in the subject. The methods of the present invention include the nasal administration of at least one vasodilator during a cardiac resuscitation procedure. A vasodilator is an agent that results in an increase in the volume of blood vessels, and results in a decrease in blood pressure in the subject. Vasodilators achieve a physiological effect that is opposite to the effect induced by a vasoconstrictor (e.g., blood vessels are expanded and blood pressure is lowered in response to a vasodilator, while blood vessels are contracted and blood pressure is raised in response to a vasoconstrictor).

The invention provides methods for improving circulation in a subject during cardiac resuscitation, reducing the risk of stroke in a subject during cardiac resuscitation, improving the efficacy of cardiac resuscitation, and providing cardiac resuscitation to a subject having cardiac arrest. These methods include nasally administering at least one dose of at least one vasodilator to the subject. Also provided are kits that include a composition containing at least one vasoconstrictor that is formulated for systemic administration and a composition containing at least one vasodilator that is formulated for nasal administration.

Provided herein are methods for improving circulation in a subject undergoing a cardiac resuscitation protocol that includes systemic administration of a vasoconstrictor that include nasally administering a therapeutically effective dose of at least one (e.g., at least two, three, or four) vasodilator to a subject undergoing a cardiac resuscitation protocol that includes systemic administration of a vasoconstrictor, where the nasal administration of the dose of the at least one vasodilator results in improved circulation in the subject as compared to control subject.

Also provided are methods of reducing the risk of stroke in a subject undergoing a cardiac resuscitation protocol that includes systemic administration of a vasoconstrictor that include nasally administering a therapeutically effective dose of at least one (e.g., at least two, three, or four) vasodilator to a subject undergoing a cardiac resuscitation protocol that includes systemic administration of a vasoconstrictor, where the nasal administration of the dose of the at least one vasodilator reduces the risk of stroke in the subject as compared to a control subject.

Also provided are methods of improving the efficacy of cardiac resuscitation in a subject undergoing a cardiac resuscitation protocol that includes systemic administration of a vasoconstrictor that include nasally administering a therapeutically effective dose of at least one (e.g., at least two, three, or four) vasodilator to a subject undergoing a cardiac resuscitation protocol that includes systemic administration of a vasoconstrictor, where the nasal administration of the dose of the at least one vasodilator improves the efficacy of cardiac resuscitation in the subject as compared to a control subject.

In some embodiments of the methods described herein, the cardiac resuscitation protocol that includes systemic administration of a vasoconstrictor includes at least one (e.g., at least two, three, four, five, six, seven, eight, nine, or ten) defibrillating shock from a charged defibrillating device. In some embodiments of the methods described herein, the cardiac resuscitation protocol that includes systemic administration of a vasoconstrictor includes at least one (e.g., at least five, ten, twenty, forty, fifty, sixty, eighty, or one hundred) chest compression.

In some embodiments of the methods described herein, the dose of the at least one vasodilator is administered to the subject within 20 minutes (e.g., within 10 minutes or within 5 minutes) of the systemic administration of the vasoconstrictor. In some embodiments of the methods described herein, the dose of the at least one vasodilator is administered to the subject no more than 20 minutes (e.g., no more than 10 minutes or no more than 5 minutes) before the systemic administration of the vasoconstrictor. In some embodiments of the methods described herein, the dose of the at least one vasodilator is administered to the subject no more than 20 minutes (e.g., no more than 10 minutes or no more than 5 minutes) after the systemic administration of the vasoconstrictor.

Also provided are methods of improving circulation in a subject undergoing a cardiac resuscitation protocol that include systemically administering a therapeutically effective dose of at least one (e.g., at least two, three, or four) vasoconstrictor to a subject undergoing a resuscitation protocol; and nasally administering a therapeutically effective dose of at least one (e.g., at least two, three, or four) vasodilator to the subject, where the systemic administration of the dose of the at least one vasoconstrictor and the nasal administration of the dose of the at least one vasodilator improves circulation in the subject as compared to a control subject.

Also provided are methods of reducing the risk of stroke in a subject undergoing a cardiac resuscitation protocol that include systemically administering a therapeutically effective dose of at least one (e.g., at least two, three, or four) vasoconstrictor to a subject undergoing a resuscitation protocol; and nasally administering a therapeutically effective dose of at least one (e.g., at least two, three, or four) vasodilator to the subject, where the systemic administration of the dose of the at least one vasoconstrictor and the nasal administration of the dose of the at least one vasodilator reduces the risk of stroke in the subject as compared to a control subject.

Also provided are methods of improving the efficacy of cardiac resuscitation in a subject undergoing a cardiac resuscitation protocol that include systemically administering a therapeutically effective dose of at least one (e.g., at least two, three, or four) vasoconstrictor to a subject undergoing a resuscitation protocol; and nasally administering a therapeutically effective dose of at least one (e.g., at least two, three, or four) vasodilator to the subject, where the systemic administration of the dose of the at least one vasoconstrictor and the nasal administration of the dose of the at least one vasodilator improves the efficacy of cardiac resuscitation in the subject as compared to a control subject.

Also provided are methods for providing cardiac resuscitation to a subject having cardiac arrest that include systemically administering a therapeutically effective dose of at least one (e.g., at least two, three, or four) vasoconstrictor to the subject; nasally administering a therapeutically effective dose of at least one (e.g., at least two, three, or four) vasodilator to the subject; and providing at least one (e.g., at least two, three, four, five, six, seven, eight, nine, or ten) defibrillating shock from a charged defibrillating device to the subject. Some embodiments further include providing at least one (e.g., at least five, ten, twenty, forty, fifty, sixty, eighty, or one hundred) chest compression to the subject. In some embodiments, the at least one defibrillating shock is provided to the subject within 20 minutes (e.g., within 10 minutes or within 5 minutes) of administering the dose of the at least one vasoconstrictor and/or within 20 minutes of administering the dose of the at least one vasodilator.

Also provided are methods for performing cardiac resuscitation in a subject having cardiac arrest that include systemically administering a therapeutically effective dose of at least one (e.g., at least two, three, or four) vasoconstrictor to the subject; nasally administering a therapeutically effective dose of at least one (e.g., at least two, three, or four) vasodilator to the subject; and providing at least one (e.g., at least five, ten, twenty, forty, fifty, sixty, eighty, or one hundred) chest compression to the subject.

In some embodiments of the methods described herein, the dose of the at least one vasoconstrictor and the dose of the at least one vasodilator are administered within 20 minutes (e.g., within 10 minutes or within 5 minutes) of each other.

In some embodiments of the methods described herein, the at least one defibrillating shock is provided to the subject no more than 20 minutes (e.g., no more than 10 minutes or no more than 5 minutes) prior to administering the dose of the at least one vasoconstrictor and/or no more than 20 minutes (e.g., no more than 10 minutes or no more than 5 minutes) prior to administering the dose of the at least one vasodilator.

In some embodiments of the methods described herein, the at least one defibrillating shock is provided to the subject no more than 20 minutes (e.g., no more than 10 minutes or no more than 5 minutes) after administering the dose of the at least one vasoconstrictor and/or no more than 20 minutes (e.g., no more than 10 minutes or no more than 5 minutes) after administering the dose of the at least one vasodilator.

In some embodiments of the methods described herein, the at least one chest compression is provided to the subject within 20 minutes (e.g., within 10 minutes or within 5 minutes) of administering the dose of the at least one vasoconstrictor and/or within 20 minutes (e.g., within 10 minutes or within 5 minutes) of administering the dose of the at least one vasodilator.

In some embodiments of the methods described herein, the at least one chest compression is provided to the subject no more than 20 minutes (e.g., no more than 10 minutes or no more than 5 minutes) prior to administering the dose of the at least one vasoconstrictor and/or no more than 20 minutes (e.g., no more than 10 minutes or no more than 5 minutes) prior to administering the dose of the at least one vasodilator.

In some embodiments of the methods described herein, chest compressions are initiated no more than 20 minutes (e.g., no more than 10 minutes or no more than 5 minutes) after administering the dose of the at least one vasoconstrictor and/or no more than 20 minutes (e.g., no more than 10 minutes or no more than 5 minutes) after administering the dose of the at least one vasodilator.

In some embodiments of the methods described herein, the at least one vasoconstrictor is administered intraperitoneally, intramuscularly, subcutaneously, intravenously, intraarterially, or intracardially.

In some embodiments of the methods described herein, the at least one vasoconstrictor is selected from the group of: tyramine, vasopressin, metaraminol, mephentermine, an amphetamine, methylphenidate, mephedrone, hydroxephedrine, angiotensin II, methoxamine, an antihistamine, oxymetazoline, propylhexedrine, caffeine, tetrahydrozoline hydrochloride, psilocybin, cocaine, lysergic acid diethylamide (LSD), lysergic acid amide (LSA), a muscarinic agonist, an adrenergic agonist, thromboxane, endothelin, asymmetric dimethylarginine, antidiuretic hormone (ADH), vasopressin, endotoxin, and thrombin.

In some embodiments of the methods described herein, the at least one vasodilator is selected from the group of: a calcium channel blocker, prostacyclin, hydralazine, minoxidil, quinapril, ramipril, a nitric oxide donor, sildenafil, tadalafil, vardenafil, tetrahydrocannabinol, theobromine, papaverine, estrogen, endothelium-derived hyperpolarizing factor (EDHF), nitric oxide (e.g., a gas containing nitric oxide), noradrenaline, histamine, prostacyclin, prostaglandin D₂, prostaglandin E₂, vasoactive intestinal peptide (VIP), L-arginine, bradykinin, substance P, niacin, platelet activating factor (PAF), a natriuretic peptide, prostaglandin I₂, prostaglandin E₂, heparin, fosinopril, lisinopril, enalapril, an alpha blocker, amyl nitrate, capsaicin, and ethanol.

In some embodiments of the methods described herein, the dose of the at least one vasodilator is formulated as a liquid. In some embodiments of any of the methods described herein, the dose of the at least one vasodilator is formulated as an aerosol.

Some embodiments of all of the methods described herein can further include selecting a subject in cardiac arrest.

Also provided are methods of using a vasodilator formulated for nasal administration in the manufacture of a medicament for improving circulation in a subject undergoing a cardiac resuscitation protocol that includes systemic administration of a vasodilator. Also provided are vasodilators formulated for nasal administration for use in improving circulation in a subject undergoing a cardiac resuscitation protocol that includes systemic administration of a vasodilator.

Also provided are methods of using a vasodilator formulated for nasal administration in the manufacture of a medicament for reducing the risk of stroke in a patient undergoing a cardiac resuscitation protocol that includes systemic administration of a vasoconstrictor. Also provided are vasodilators formulated for nasal administration for use in reducing the risk of stroke in a patient undergoing a cardiac resuscitation protocol that includes systemic administration of a vasoconstrictor.

Also provided are methods of using a vasodilator formulated for nasal administration in the manufacture of a medicament for improving the efficacy of a cardiac resuscitation protocol in a subject undergoing a cardiac resuscitation protocol that includes systemic administration of a vasoconstrictor. Also provided are vasodilators formulated for nasal administration for use in improving the efficacy of a cardiac resuscitation protocol in a subject undergoing a cardiac resuscitation protocol that includes systemic administration of a vasoconstrictor.

Also provided are methods of using a vasodilator formulated for nasal administration and a vasoconstrictor formulated for systemic administration in the manufacture of a kit for improving circulation in a subject undergoing a cardiac resuscitation protocol. Also provided are vasodilators formulated for nasal administration and vasoconstrictors formulated for systemic administration for use in improving circulation in a subject undergoing a cardiac resuscitation protocol.

Also provided are methods of using a vasodilator formulated for nasal administration and a vasoconstrictor formulated for systemic administration in the manufacture of a kit for reducing the risk of stroke in a subject undergoing a cardiac resuscitation protocol. Also provided are vasodilators formulated for nasal administration and vasoconstrictors formulated for systemic administration for use in reducing the risk of stroke in a subject undergoing a cardiac resuscitation protocol.

Also provided are methods of using a vasodilator formulated for nasal administration and a vasoconstrictor formulated for systemic administration in the manufacture of a kit for improving the efficacy of cardiac resuscitation in a subject undergoing a cardiac resuscitation protocol. Also provided are vasodilators formulated for nasal administration and vasoconstrictors formulated for systemic administration for use in improving the efficacy of cardiac resuscitation in a subject undergoing a cardiac resuscitation protocol.

Also provided are methods of using a vasodilator formulated for nasal administration, a vasoconstrictor formulated for systemic administration, and a defibrillator in the manufacture of a kit for performing cardiac resuscitation in a subject having cardiac arrest. Also provided are vasodilators formulated for nasal administration, vasoconstrictors formulated for systemic administration, and a defibrillator for use in performing cardiac resuscitation in a subject having cardiac arrest.

Also provided are kits containing a composition containing at least one vasoconstrictor (e.g., any of the vasoconstrictors described herein or known in the art) that is formulated for systemic administration, and a composition containing at least one vasodilator (e.g., any of the vasodilators described herein or known in the art) that is formulated for nasal administration. In some embodiments of the kits described herein, the composition containing at least one vasodilator is formulated as a liquid. In some embodiments of the kits described herein, the composition containing at least one vasodilator is formulated as an aerosol. Some embodiments of the kits described herein further contain a nebulizer that contains the aerosol.

In some embodiments of the kits described herein, the composition containing at least one vasoconstrictor (e.g., any of the vasoconstrictors described herein or known in the art) is formulated in a therapeutically effective dose. In some embodiments of the kits described herein, the composition containing at least one vasodilator is formulated in a therapeutically effective dose. Some embodiments of the kits described herein further contain a defibrillating device.

In general, cardiac arrest is the cessation of normal circulation of the blood in a subject due to failure of the heart to contract effectively. Cardiac arrest can result from any number of causes. Non-limiting causes of cardiac arrest include: heart attack, suffocation, hypothermia, anaphylaxis, hemorrhage, poisoning, heart failure, and trauma. Additional causes of cardiac arrest are known in the art.

In general, a defibrillating shock relates to the administration of a therapeutically effective voltage to a subject in order to stimulate the heart to contract effectively (e.g., restart heart contractions) in a subject. Methods of providing a defibrillating shock to a subject (e.g., a subject having cardiac arrest) are known in the art.

In general, a defibrillating device can be any instrument or medical device that can be used to provide a defibrillating shock to a subject (e.g., a subject having cardiac arrest). A variety of defibrillating devices are commercially available.

In general, improving circulation in a subject undergoing a cardiac resuscitation protocol results in an increase (e.g., a detectable, observable, or significant increase) in the blood flow to one or more (e.g., one, two, three, or four) vital organs (e.g., heart, lungs, brain, kidneys, liver, and pancreas) in a subject, following a period of cardiac arrest in a subject that has received or is receiving a cardiac resuscitation protocol, as compared to the amount of blood flow to the same one or more vital organs in a control subject (e.g., a control not receiving the cardiac resuscitation protocol or receiving a different cardiac resuscitation protocol) or in the same subject prior to receiving the cardiac resuscitation protocol.

By the term “improving” or “increase” is meant an observable, detectable, and/or significant increase compared to a control.

In general, a stroke is a disorder characterized by the loss of brain function due to disturbance (e.g., cessation) of blood supply to brain tissue. Exemplary methods for diagnosing stroke and the symptoms of stroke are known in the art. Methods for reducing the risk of having a stroke in a subject undergoing a cardiac resuscitation protocol are provided herein.

In general, reducing the risk of stroke results in a decrease (e.g., a significant decrease) in the risk of having a stroke in a subject that has received or is receiving a specific cardiac resuscitation protocol compared a control subject or population (e.g., a control subject or population having cardiac arrest but receiving a different cardiac resuscitation protocol or receiving no cardiac resuscitation protocol).

By the term “control subject” or “control population” is meant a subject (or patient population) having cardiac arrest that is treated with an alternate cardiac resuscitation protocol, or a subject (or patient population) having cardiac arrest that does not receive a cardiac resuscitation protocol. In some embodiments, a control subject or patient population can be matched for one or more biological parameters (e.g., matched for one or more of age, weight, sex, cause of cardiac arrest, prior history of a specific cardiovascular disease, and prior history of stroke).

In general, a chest compression is the application of force to an area of the chest proximal to (e.g., directly above) the heart of a subject. In non-limiting examples, the application of force can result in a depression of the chest tissue of the subject (e.g., an approximately 5-cm depression in the chest tissue of the subject). Exemplary methods of providing one or more chest compressions to a subject are known in the art.

In general, cardiac resuscitation or cardiac resuscitation protocol is a therapeutic treatment performed in order to increase circulation of the blood or restore circulation of the blood in a subject having heart failure. Non-limiting examples of cardiac resuscitation are known in the art, and include cardiac pulmonary resuscitation (CPR) and providing a defibrillating shock using a defibrillating device. Additional methods for performing cardiac resuscitation are described herein.

In general, a cardiac resuscitation protocol that includes systemic administration of a vasoconstrictor is a cardiac resuscitation protocol that includes the systemic (e.g., subcutaneous, intravenous, intraarterial, intraperitoneal, intracardiac, or intramuscular) administration of at least one (e.g., at least two, three, four, or five) dose of at least one (e.g., at least two, three, four, or five) vasoconstrictor.

In general, a vasoconstrictor is a pharmaceutical agent that results in the narrowing of a blood vessel (e.g., a large artery or a small arteriole) in a mammal (e.g., a human). Vasoconstrictors are also referred to as vasopressors or pressors by medical professionals. A variety of vasoconstrictors are known in the art. Non-limiting examples of vasoconstrictors are also described herein. Any of the exemplary vasoconstrictors described herein or vasoconstrictors known in the art can be used in any of the methods, compositions, or kits described herein.

In general, a vasodilator is a pharmaceutical agent that results in the widening of a blood vessel (e.g., a large artery or a small arteriole) in a subject (e.g., a human). A variety of vasodilators are known in the art. Non-limiting examples of vasodilators are also described herein. Any of the exemplary vasodilators described herein or vasodilators known in the art can be used in any of the methods, compositions, or kits described herein.

By the term “a subject” is meant any mammal. In some embodiments, the mammal is a human (e.g., a female, a male, an adult, a child, a teenager, or an infant), a mouse, a rat, a pig, a guinea pig, or a dog.

By the phrase “systemic administration” or “systemically administering” is meant the non-specific delivery of at least one therapeutic agent to the majority of the tissues in the body of a mammal (e.g., a human). Non-limiting examples of systemic administration include intravenous, intraarterial, intracardiac, subcutaneous, intramuscular, and intraperitoneal administration.

By the phrase “nasal administration” or “nasally administering” is meant the delivery of at least one therapeutic agent to the nasal or sinus mucosal tissue of a subject (e.g., a human). As is known in the art, delivery of a therapeutic agent to the nasal or sinus tissue of a subject can result in the successful delivery of the therapeutic agent to the brain tissue and/or the synovial fluid of the subject.

The phrase “therapeutically effective dose” refers generally to an amount necessary or sufficient to bring about a desired biological result or treatment outcome. For example, a therapeutically effective amount of a vasoconstrictor can be an amount sufficient to stimulate a narrowing of a blood vessel in a tissue within a subject (e.g., a human subject). In another example, a therapeutically effective amount of a vasodilator can be an amount sufficient to stimulate a widening of a blood vessel in a tissue within a subject (e.g., widening or dilation of a blood vessel within the brain of a human subject).

An increase in the efficacy of cardiac resuscitation is a relative increase in the success of a cardiac resuscitation protocol in a subject having cardiac arrest (e.g., a human subject) compared to a control subject or population having cardiac arrest but receiving a different cardiac resuscitation protocol or not receiving a cardiac resuscitation protocol. In non-limiting examples, the efficacy of a cardiac resuscitation protocol may be assessed by following one or more of the following: restoration of blood flow to one or more vital organs in the mammal, improvement of blood flow to the subject's extremities, a decrease in ischemic-reperfusion injury in one or more tissues (e.g., one or more vital organs) in the mammal following cardiac arrest, and the mammal's survival of cardiac arrest. Additional parameters useful for the assessment of the efficacy of a cardiac resuscitation protocol are described herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an exemplary example of a person providing a defibrillating shock from a charged defibrillating device to a subject having cardiac arrest.

### DETAILED DESCRIPTION

The invention provides methods for improving circulation in a subject during cardiac resuscitation, reducing the risk of stroke in a subject during or after cardiac resuscitation, and methods of improving the efficacy of cardiac resuscitation. Also provided are methods of providing cardiac resuscitation to a subject having cardiac arrest, and kits including a composition that contains at least one vasoconstrictor that is formulated for systemic administration, and a composition that contains at least one vasodilator that is formulated for nasal administration.

### Cardiac Arrest

Cardiac arrest is cessation of normal circulation of the blood in a subject due to failure of the heart to contract effectively. Cardiac arrest can result from any number of causes, including but not limited to heart attack, suffocation, hypothermia, anaphylaxis, hemorrhage, poisoning, heart failure, trauma, coronary artery disease, cardiomyopathy, valvular heart disease, congenital heart disease, and electrical problems in the heart (e.g., primary heart rhythm abnormalities, such as Brugada's syndrome and long QT syndrome).

Symptoms of cardiac arrest include sudden collapse, no pulse, no breathing, and loss of consciousness. In some instances, other signs and symptoms precede cardiac arrest including fatigue, fainting, blackouts, dizziness, chest pain, shortness of breath, weakness, palpitations, and/or vomiting. In some instances, cardiac arrest occurs with no warning.

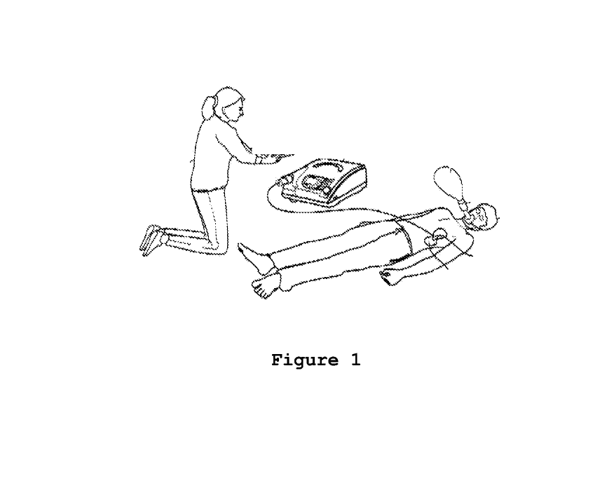

Subjects having cardiac arrest often receive emergency cardiac resuscitation. The most common forms of cardiac resuscitation (cardiac resuscitation protocols) performed on a subject having cardiac arrest include cardiopulmonary resuscitation (CPR) and/or providing at least one defibrillating shock from a charged defibrillating device (e.g., a portable defibrillator) to the subject (see, e.g., FIG. 1). Cardiac resuscitation protocols can be performed by any person, including health care professionals (e.g., an emergency medical technician (EMT), a nurse, a physician's assistant, a nurse's assistant, or a physician) or other emergency response personnel (e.g., a firefighter, a security guard, a medical emergency response officer, or a police officer). Cardiac resuscitation protocols can be performed at any location (e.g., at a health care facility, in an ambulance, or at the subject's personal residence, workplace, or any other location).

In some examples, during a cardiac resuscitation protocol (e.g., CPR) it can be beneficial to administer a therapeutic dose of a vasoconstrictor to counteract a dilation of blood vessels which can occur during cardiac arrest. Administration of the vasoconstrictor results in a narrowing of the blood vessels which can improve the effectiveness of a cardiac resuscitation protocol. However, when a vasoconstrictor is delivered systemically, it constricts the blood vessels in the brain in addition to other blood vessels in the patient's body. This constriction of blood vessels in the brain can make it difficult to circulate blood to this vital organ. As described in more detail herein, the concurrent or near concurrent administration of a vasodilator in addition to the vasoconstrictor (e.g., administration of the vasodilator within about 5 minutes of administration of the vasoconstrictor, administration of the vasodilator within about 3 minutes of administration of the vasoconstrictor, administration of the vasodilator within about 1 minute of administration of the vasoconstrictor) can dilate specific sets of blood vessels to provide preferential perfusion into regions targeted by the vasoconstrictor. Thus, in the examples described herein, a vasoconstrictor is administered to a patient on a systemic basis and a vasoconstrictor is administered to a patient on a targeted basis (e.g., by administration in the membrane of the nasal cavity for targeted application to the blood vessels in the brain).

### Vasoconstrictors

Vasoconstrictors are pharmaceutical agents that result in the narrowing of a blood vessel (e.g., a large artery or a small arteriole) in a mammal (e.g., a human) (when administered in a therapeutically effective dose). In some of the methods described herein, at least dose of at least one (e.g., at least two, three, four, five, or six) vasoconstrictor can be systemically administered (e.g., intravenous, intaarterial, subcutaneous, intramuscular, subcutaneous, intraperitoneal, or intracardiac administration) to a subject.

Non-limiting examples of vasoconstrictors include tyramine, vasopressin, metaraminol, mephentermine, amphetamines (e.g., methamphetamine and hydroxyamphetamine), methylphenidate, mephedrone, hydroxephedrine, angiotensin II, methoxamine, antihistamines, oxymetazoline, propylhexedrine, stimulants, caffeine, tetrahydrozoline hydrochloride, psilocybin, cocaine, lysergic acid diethylamide (LSD), lysergic acid amide (LSA), muscarinic agonists (e.g., acetylcholine), adrenergic agonists (e.g., epinephrine, norepinephrine, ephedrine, phenylephrine, pseudoephedrine, and dopamine), thromboxane, endothelin, asymmetric dimethylarginine, antidiuretic hormone (ADH), vasopressin, endotoxin, and thrombin. Additional examples of vasoconstrictors are known in the art. One or more (e.g., at least two, three, or four) of any of the exemplary vasoconstrictors described herein and/or one or more (e.g., at least two, three, or four) of any vasoconstrictors known in the art can be used in any of the methods, compositions, and kits described herein without limitation.

In some of the methods described herein, at least one (e.g., at least two, three, four, five, or six) dose of at least one (e.g., at least two, three, four, five, or six) vasoconstrictor can be systemically administered to a subject having cardiac arrest in a dose (for each vasoconstrictor present in the dose) of anywhere between 10 μg to 500 mg, e.g., 100 μg to 400 mg, 100 μg to 350 mg, 100 μg to 300 mg, 100 μg to 250 mg, 100 μg to 200 mg, 100 μg to 150 mg, 1 mg to 200 mg, 5 mg to 200 mg, 5 mg to 150 mg, 5 mg to 100 mg, 10 mg to 100 mg, 50 to 100 mg, 1 mg to 50 mg, or 1 mg to 25 mg, or any other dose for systemic administration of a vasoconstrictor known in the art. In some embodiments of the methods described herein, the at least one dose of the at least one vasoconstrictor can be systemically administered to the subject within 1 hour of the onset of cardiac arrest (e.g., within 45, 30, 20, 15, 10, or 5 minutes of the onset of cardiac arrest). In some embodiments where two or more doses (e.g., at least three, four, or five doses) are administered to the subject, at least two temporal administrations of at least one vasoconstrictor are spaced at least 1 minute (e.g., at least 5, 10, 15, 20, 25, 30, 35, 40, 45, or 50 minutes, or at least one hour) apart.

In some embodiments of the methods described herein, the at least one dose of at least one vasoconstrictor is systemically administered within one hour (within 55, 50, 45, 40, 35, 30, 25, 20, 15, 10, 5, or 1 minute(s)) of at least one chest compression or within one hour (within 55, 50, 45, 40, 35, 30, 25, 20, 15, 10, 5, or 1 minute(s)) of providing a defibrillating shock from a charged defibrillating device. In some embodiments of any of the methods described herein, the at least one dose of at least one vasoconstrictor is systemically administered no more than one hour (e.g., no more than 55, 50, 45, 40, 35, 30, 25, 20, 15, 10, 5, or 1 minute(s)) before administration of at least one chest compression, or systemically administered no more than one hour (e.g., no more than 55, 50, 45, 40, 35, 30, 25, 20, 15, 10, 5, or 1 minute(s)) before providing a defibrillating shock from a charged defibrillating device. In some embodiments of any of the methods described herein, the at least one dose of at least one vasoconstrictor is systemically administered no later than one hour (e.g., no later than 55, 50, 40, 35, 30, 25, 20, 15, 10, 5, or 1 minute(s)) after the initiation of chest compressions, or no later than one hour (e.g., no later than 55, 50, 45, 40, 35, 30, 25, 20, 15, 10, 5, or 1 minute(s)) after providing a first defibrillating shock to the subject from a charged defibrillating device.

In some embodiments of the methods described herein, a subject is nasally administered at least one dose of at least one vasodilator within one hour (e.g., within 55, 50, 45, 40, 35, 30, 25, 20, 15, 10, 5, or 1 minute(s)) of administration of a dose of at least one vasoconstrictor. In some embodiments, the at least one dose of at least one vasodilator is nasally administered no more than one hour before (e.g., no more than 55, 50, 45, 40, 35, 30, 25, 20, 15, 10, 5, or 1 minute(s) before) systemic administration of a dose of at least one vasoconstrictor. In some embodiments, the at least one dose of at least one vasodilator is nasally administered no more than one hour after (e.g., no more than 55, 50, 45, 40, 35, 30, 25, 20, 15, 10, 5, or 1 minute(s) after) systemic administration of a dose of at least one vasoconstrictor. In some embodiments, at least one dose of at least one vasodilator is administered at substantially the same time (e.g., within two minutes or within 1 minute) as the systemic administration of a dose of at least one vasoconstrictor.

### Vasodilators

Vasodilators are pharmaceutical agents that result in the widening of a blood vessel (e.g., a large artery or a small arteriole) in a subject (e.g., a human). As vasoconstrictors are typically administered to a subject having cardiac arrest, the methods described herein differ from the present standard of care for subjects having cardiac arrest. In the methods described herein, at least one dose of at least one vasodilator is nasally administered to a subject.

Non-limiting examples of vasodilators include calcium channel blockers (e.g., adenosine), prostacyclin, hydralazine, minoxidil, quinapril, ramipril, a nitric oxide donor (e.g., nitroglycerin, isosorbide mononitrate, isosorbide dinitrate, pentaerythrital tetranitrate, and sodium nitroprusside), sildenafil, tadalafil, vardenafil, tetrahydrocannabinol, theobromine, papaverine, estrogen, endothelium-derived hyperpolarizing factor (EDHF), nitric oxide (e.g., a gas containing nitric oxide), noradrenaline, histamine, prostacyclin, prostaglandin D₂, prostaglandin E₂, vasoactive intestinal peptide (VIP), L-arginine, bradykinin, substance P, niacin, platelet activating factor (PAF), natriuretic peptides (e.g., atrial natriuretic peptide (ANP) or brain natriuretic peptide (BNP)), prostaglandin I₂, prostaglandin E₂, heparin, fosinopril, lisinopril, enalapril, an alpha blocker, amyl nitrate, capsaicin, and ethanol. Additional examples of vasodilators are known in the art. One or more (e.g., at least two, three, or four) of any of the exemplary vasodilators described herein and/or one or more (e.g., at least two, three, or four) of any vasoconstrictors known in the art can be used in any of the methods, compositions, and kits described herein without limitation.

In some of the methods described herein, at least one (e.g., at least two, three, four, five, or six) dose of at least one (e.g., at least two, three, four, five, or six) vasodilator can be nasally administered to a subject having cardiac arrest in a dose (for each vasodilator present in the dose) of anywhere between 10 μg to 500 mg, e.g., 100 μg to 400 mg, 100 μg to 350 mg, 100 μg to 300 mg, 100 μg to 250 mg, 100 μg to 200 mg, 100 μg to 150 mg, 1 mg to 200 mg, 5 mg to 200 mg, 5 mg to 150 mg, 5 mg to 100 mg, 10 mg to 100 mg, 50 to 100 mg, 1 mg to 50 mg, or 1 mg to 25 mg, or any other dose for nasal administration of a vasodilator known in the art. In some embodiments of the methods described herein, the at least one dose of the at least one vasodilator can be nasally administered to the subject within 1 hour of the onset of cardiac arrest (e.g., within 55, 50, 45, 40, 35, 30, 35, 20, 15, 10, 5, or 1 minute(s)). In some embodiments where two or more doses (e.g., at least three, four, five, or six doses) are administered to the subject, at least two temporal administrations of at least one vasodilator are spaced at least 1 minute (e.g., at least 2, 5, 10, 15, 20, 25, 30, 35, 40, 45, 50, or 55 minutes, or at least one hour) apart.

In some embodiments of the methods described herein, the at least one dose of at least one vasodilator is nasally administered within one hour (within 55, 50, 45, 40, 35, 30, 25, 20, 15, 10, 5, or 1 minute(s)) of at least one chest compression or within one hour (within 55, 50, 45, 40, 35, 30, 25, 20, 15, 10, 5, or 1 minute(s)) of providing a defibrillating shock from a charged defibrillating device. In some embodiments of any of the methods described herein, the at least one dose of at least one vasodilator is nasally administered no more than one hour (e.g., no more than 55, 50, 45, 40, 35, 30, 25, 20, 15, 10, 5, or 1 minute(s)) before administration of at least one chest compression, or nasally administered no more than one hour (e.g., no more than 55, 50, 45, 40, 35, 30, 25, 20, 15, 10, 5, or 1 minute(s)) before providing at least one defibrillating shock from a charged defibrillating device. In some embodiments of any of the methods described herein, the at least one dose of at least one vasodilator is nasally administered no later than one hour (e.g., no later than 55, 50, 45, 40, 35, 30, 25, 20, 15, 10, 5, or 1 minute(s)) after initiation of chest compressions, or no later than one hour (e.g., no later than 55, 50, 45, 40, 35, 30, 25, 20, 15, 10, 5, or 1 minute(s)) after providing a first defibrillating shock from a charged defibrillating device.

In some embodiments of the methods described herein, a subject is systemically administered at least one dose of at least one vasoconstrictor within one hour (e.g., within 55, 50, 45, 40, 35, 30, 25, 20, 15, 10, 5, or 1 minute(s)) of nasal administration of a dose of at least one vasodilator. In some embodiments, the at least one dose of at least one vasoconstrictor is systemically administered no more than one hour before (e.g., no more than 55, 50, 45, 40, 35, 30, 25, 20, 15, 10, 5, or 1 minute(s) before) nasal administration of a dose of at least one vasodilator. In some embodiments, the at least one dose of at least one vasoconstrictor is systemically administered no more than one hour after (e.g., no more than 55, 50, 45, 40, 35, 30, 25, 20, 15, 10, 5, or 1 minute(s) after) nasal administration of a dose of at least one vasodilator.

### Methods for Improving Circulation in a Subject During Cardiac Resuscitation

Provided herein are methods for improving circulation (e.g., a significant, detectable, or observable improvement in circulation) in a subject undergoing a cardiac resuscitation protocol that includes systemic administration of a vasoconstrictor that include nasally administering a therapeutically effective dose of at least one vasodilator to a subject undergoing a cardiac resuscitation protocol that includes systemic administration of at least one vasoconstrictor, where the nasal administration of the dose of the at least one vasodilator results in improved circulation in the subject as compared to a control subject (e.g., a subject undergoing a cardiac resuscitation protocol that (i) includes systemic administration of at least one vasoconstrictor and (ii) does not include nasal administration of a vasodilator). In some embodiments, the cardiac resuscitation protocol that includes systemic administration of a vasoconstrictor includes at least one defibrillating shock from a charged defibrillating device and/or includes at least one chest compression. In some embodiments, the dose of the at least one vasodilator is nasally administered to the subject within one hour (e.g., within 55, 50, 45, 40, 35, 30, 25, 20, 15, 10, 5, or 1 minute(s)) of the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) before the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) after the systemic administration of the vasoconstrictor. In some embodiments, the dose of the at least one vasodilator is nasally administered to the subject at substantially the same time (e.g., within 2 minutes or within 1 minute) as the systemic administration of the vasoconstrictor.

Also provided are methods of improving circulation (e.g., a significant, detectable, or observable improvement in circulation) in a subject undergoing a cardiac resuscitation protocol that include systemically administering a therapeutically effective dose of at least one (e.g., at least two, three, four, five, or six) vasoconstrictor to a subject undergoing a resuscitation protocol; and nasally administering a therapeutically effective dose of at least one (e.g., at least two, three, four, five, or six) vasodilator to the subject, wherein the systemic administration of the dose of the at least one vasoconstrictor and the nasal administration of the dose of the at least one vasodilator improves circulation in the subject as compared to a control subject (e.g., a subject undergoing a resuscitation protocol that (i) does not include systemic administration of a vasoconstrictor and/or (ii) does not include nasal administration of a vasodilator). In some embodiments, the dose of the at least one vasoconstrictor and the dose of the at least one vasodilator are administered within one hour (e.g., within 55, 50, 45, 40, 35, 30, 25, 20, 15, 10, 5, or 1 minute(s)) of each other.

In some embodiments, the dose of the at least one vasoconstrictor is systemically administered to the subject before the nasal administration of the dose of the at least one vasodilator. In some embodiments, the dose of the at least one vasodilator is nasally administered to the subject before the systemic administration of the dose of the at least one vasoconstrictor. In some embodiments, the dose of the at least one vasoconstrictor is systemically administered to the subject and the dose of the at least one vasodilator is nasally administered to the subject at approximately the same time (e.g., within 2 minutes or within 1 minute of each other).

In some embodiments, the dose of the at least one vasodilator is nasally administered and/or the dose of the at least one vasoconstrictor is systemically administered prior to the delivery of a chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of a chest compression to the subject). In some embodiments, the dose of the at least one vasodilator is nasally administered and/or the dose of the at least one vasoconstrictor is systemically administered after the delivery of a chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after delivery of a chest compression to the subject). In some embodiments, the dose of the at least one vasoconstrictor is systemically administered to the subject and the dose of the at least one vasodilator is nasally administered to the subject at approximately the same time as the delivery of the chest compression (e.g., within 2 minutes or within 1 minute of each other).

In some embodiments, the dose of the at least one vasodilator is nasally administered and/or the dose of the at least one vasoconstrictor is systemically administered prior to the delivery of a defibrillating shock using a charged defibrillation device (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of a defibrillating shock to the subject). In some embodiments, the dose of the at least one vasodilator is nasally administered and/or the dose of the at least one vasoconstrictor is systemically administered after the delivery of a first defibrillating shock (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after delivery of a first defibrillating shock to the subject). In some embodiments, the dose of the at least one vasoconstrictor is systemically administered to the subject and the dose of the at least one vasodilator is nasally administered to the subject at approximately the same time as the delivery of the defibrillating shock (e.g., within 2 minutes or within 1 minute of each other).

In some embodiments, the dose of the at least one vasodilator is nasally administered and/or the dose of the at least one vasoconstrictor is systemically administered prior to the delivery of a defibrillating shock using a charged defibrillation device and prior to the performance of a chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of the later of the defibrillating shock and the chest compression to the subject). In some embodiments, the dose of the at least one vasodilator is nasally administered and/or the dose of the at least one vasoconstrictor is systemically administered after the delivery of a first defibrillating shock and the initiation of chest compressions to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after the later of the delivery of the defibrillating shock or the delivery of the defibrillating shock to the subject). In some embodiments, the dose of the at least one vasoconstrictor is systemically administered to the subject and the dose of the at least one vasodilator is nasally administered to the subject at approximately the same time as the delivery of the chest compression and the defibrillating shock (e.g., within 2 minutes or within 1 minute of each other).

The dose of the at least one (e.g., at least two, three, four, five, or six) vasodilator can contain any of the exemplary vasodilators described herein or known in the art, in any combination. The dose of the at least one vasodilator nasally administered to the subject can be any of the doses described herein or known in the art. The at least one vasodilator can be formulated for nasal administration using any of the techniques described herein or known in the art (e.g., an aerosol or a liquid formulation).

The dose of the at least one (e.g., at least two, three, four, five, or six) vasoconstrictors can contain any of the exemplary vasoconstrictors described herein or known in the art in any combination. The dose of the at least one vasoconstrictor systemically administered to the subject can be any of the doses described herein or known in the art. The at least one vasoconstrictor can be formulated for systemic administration using any of the techniques described herein or known in the art (e.g., a liquid formulation). In some embodiments, the systemic administration is intravenous, intaarterial, subcutaneous, intramuscular, subcutaneous, intraperitoneal, or intracardiac administration.

The methods described herein can include the systemic administration of one or more vasoconstrictors (e.g., any of the exemplary vasoconstrictors described herein and/or known in the art) and the nasal administration of one or more vasodilators (e.g., any of the exemplary vasodilators described herein and/or known in the art) in any combination without limitation.

An improvement in circulation of a subject (e.g., during or after a cardiac resuscitation procedure) can be assessed by a health care professional or emergency medical personnel by following a number of different physiological parameters, e.g., blood flow to one or more tissues in the body of the subject (e.g., blood flow to one or more of the vital organs of the subject, or blood flow to the subject's extremities). Methods for detecting blood flow to one or more tissues in the body of the subject are known in the art, and include, e.g., ankle brachial index, detected pulse in the extremities, detected pulse in the carotid artery, and observing skin coloration.

An observed or detected improvement can be relative to the circulation in the same subject having cardiac arrest prior to cardiac resuscitation or a control subject having cardiac arrest, but receiving a different treatment (e.g., a cardiac resuscitation protocol that does not include nasal administration of a vasodilator) or no treatment.

### Methods of Reducing the Risk of Stroke in a Subject During Cardiac Resuscitation

Stroke is a negative side effect of subjects that receive or have received cardiac resuscitation. The invention also provides methods of reducing the risk (e.g., a significant decrease) of stroke in a subject undergoing a cardiac resuscitation protocol that includes systemic administration of a vasoconstrictor, that include nasally administering a therapeutically effective dose of at least one (e.g., at least two, three, four, five, or six) vasodilator to a subject undergoing a cardiac resuscitation protocol that includes systemic administration of a vasoconstrictor, where the nasal administration of the dose of the at least one vasodilator reduces the risk of stroke in the subject as compared to a control subject (e.g., a subject undergoing a cardiac resuscitation protocol that (i) includes systemic administration of a vasoconstrictor and (ii) does not include nasal administration of a vasodilator). In some embodiments, the cardiac resuscitation protocol that includes systemic administration of a vasoconstrictor includes at least one defibrillating shock from a charged defibrillating device and/or includes at least one chest compression. In some embodiments, the dose of the at least one vasodilator is nasally administered to the subject within one hour (e.g., within 55, 50, 45, 40, 35, 30, 25, 20, 15, 10, 5, or 1 minute(s)) of the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) before the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) after the systemic administration of the vasoconstrictor.

Also provided are methods of reducing the risk of stroke in a subject undergoing a cardiac resuscitation protocol that include systemically administering a therapeutically effective dose of at least one (e.g., at least two, three, four, five, or six) vasoconstrictor to a subject undergoing a resuscitation protocol; and nasally administering a therapeutically effective dose of at least one (e.g., at least two, three, four, five, or six) vasodilator to the subject, where the systemic administration of the dose of the at least one vasoconstrictor and the nasal administration of the dose of the at least one vasodilator reduces the risk of stroke in the subject as compared to a control subject (e.g., a subject undergoing a resuscitation protocol that (i) does not include systemic administration of a vasoconstrictor and/or (ii) does not include nasal administration of a vasodilator). In some embodiments, the dose of the at least one vasoconstrictor and the dose of the at least one vasodilator are administered within one hour (e.g., within 55, 50, 45, 40, 35, 30, 25, 20, 15, 10, 5, or 1 minute(s)) of each other.

In some embodiments, the dose of the at least one vasoconstrictor is systemically administered to the subject before the nasal administration of the dose of the at least one vasodilator. In some embodiments, the dose of the at least one vasodilator is nasally administered to the subject before the systemic administration of the dose of the at least one vasoconstrictor. In some embodiments, the dose of the at least one vasoconstrictor is systemically administered to the subject and the dose of the at least one vasodilator is nasally administered to the subject at approximately the same time (e.g., within 2 minutes or within 1 minute of each other).

In some embodiments, the dose of the at least one vasodilator is nasally administered and/or the dose of the at least one vasoconstrictor is systemically administered prior to the delivery of a chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of a chest compression to the subject). In some embodiments, the dose of the at least one vasodilator is nasally administered and/or the dose of the at least one vasoconstrictor is systemically administered after the initiation of chest compressions to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after the initiation of chest compressions to the subject). In some embodiments, the dose of the at least one vasoconstrictor is systemically administered to the subject and the dose of the at least one vasodilator is nasally administered to the subject at approximately the same time as the delivery of the chest compression (e.g., within 2 minutes or within 1 minute of each other).

In some embodiments, the dose of the at least one vasodilator is nasally administered and/or the dose of the at least one vasoconstrictor is systemically administered prior to the delivery of a defibrillating shock using a charged defibrillation device (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of a defibrillating shock to the subject). In some embodiments, the dose of the at least one vasodilator is nasally administered and/or the dose of the at least one vasoconstrictor is systemically administered after the delivery of a first defibrillating shock to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after delivery of a first defibrillating shock to the subject). In some embodiments, the dose of the at least one vasoconstrictor is systemically administered to the subject and the dose of the at least one vasodilator is nasally administered to the subject at approximately the same time as the delivery of the defibrillating shock (e.g., within 2 minutes or within 1 minute of each other).

In some embodiments, the dose of the at least one vasodilator is nasally administered and/or the dose of the at least one vasoconstrictor is systemically administered prior to the delivery of a defibrillating shock using a charged defibrillation device and prior to the performance of a chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of the later of the defibrillating shock and the chest compression to the subject). In some embodiments, the dose of the at least one vasodilator is nasally administered and/or the dose of the at least one vasoconstrictor is systemically administered after the delivery of a first defibrillating shock and after initiation of chest compressions to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after the later of the delivery of the first defibrillating shock or the initiation of chest compressions). In some embodiments, the dose of the at least one vasoconstrictor is systemically administered to the subject and the dose of the at least one vasodilator is nasally administered to the subject at approximately the same time as the delivery of the chest compression and the defibrillating shock (e.g., within 2 minutes or within 1 minute of each other).

The dose of the at least one (e.g., at least two, three, four, five, or six) vasodilator can contain any of the exemplary vasodilators described herein or known in the art in any combination. The dose of the at least one vasodilator nasally administered to the subject can be any of the doses described herein or known in the art. The at least one vasodilator can be formulated for nasal administration using any of the techniques described herein or known in the art (e.g., an aerosol or a liquid formulation).

The dose of the at least one (e.g., at least two, three, four, five, or six) vasoconstrictors can contain any of the exemplary vasoconstrictors described herein or known in the art in any combination. The dose of the at least one vasoconstrictor systemically administered to the subject can be any of the doses described herein or known in the art. The at least one vasoconstrictor can be formulated for systemic administration using any of the techniques described herein or known in the art (e.g., a liquid formulation). In some embodiments, the systemic administration is intravenous, intaarterial, subcutaneous, intramuscular, subcutaneous, intraperitoneal, or intracardiac administration.

The methods described herein can include the systemic administration of one or more vasoconstrictors (e.g., any of the exemplary vasoconstrictors described herein and/or known in the art) and the nasal administration of one or more vasodilators (e.g., any of the exemplary vasodilators described herein and/or known in the art) in any combination without limitation.

### Methods of Improving the Efficacy of Cardiac Resuscitation in a Subject

Also provided are methods of improving (e.g., a significant, detectable, or observable improvement) the efficacy of cardiac resuscitation in a subject undergoing a cardiac resuscitation protocol that includes systemic administration of a vasoconstrictor, that include nasally administering a therapeutically effective dose of at least one (e.g., at least two, three, four, five, or six) vasodilator to a subject undergoing a cardiac resuscitation protocol that includes systemic administration of a vasoconstrictor, where the nasal administration of the dose of the at least one vasodilator improves the efficacy of cardiac resuscitation in the subject as compared to a control subject (e.g., a subject undergoing a cardiac resuscitation protocol that (i) includes systemic administration of a vasoconstrictor and (ii) does not include nasal administration of a vasodilator). In some embodiments, the cardiac resuscitation protocol that includes systemic administration of a vasoconstrictor includes at least one (e.g., at least two, three, four, or five) defibrillating shock from a charged defibrillating device and/or includes at least one (e.g., at least 5, 10, 15, 20, 25, 50, or 100) chest compression. In some embodiments, the dose of the at least one vasodilator is nasally administered to the subject within one hour (e.g., within 55, 50, 45, 40, 35, 30, 25, 20, 15, 10, 5, or 1 minute(s)) of the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) before the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) after the systemic administration of the vasoconstrictor.

Also provided are methods of improving the efficacy of cardiac resuscitation in a subject undergoing a cardiac resuscitation protocol that include systemically administering a therapeutically effective dose of at least one (e.g., at least two, three, four, five, or six) vasoconstrictor to a subject undergoing a resuscitation protocol; and nasally administering a therapeutically effective dose of at least one (e.g., at least two, three, four, five, or six) vasodilator to the subject, where the systemic administration of the dose of the at least one vasoconstrictor and the nasal administration of the dose of the at least one vasodilator improves the efficacy of cardiac resuscitation in the subject as compared to a control subject (e.g., a subject undergoing a resuscitation protocol that (i) does not include systemic administration of a vasoconstrictor and/or (ii) does not include nasal administration of a vasodilator). In some embodiments, the dose of the at least one vasoconstrictor and the dose of the at least one vasodilator are administered within one hour (e.g., within 55, 50, 45, 40, 35, 30, 25, 20, 15, 10, 5, or 1 minute(s)) of each other.

In some embodiments, the dose of the at least one vasoconstrictor is systemically administered to the subject before the nasal administration of the dose of the at least one vasodilator. In some embodiments, the dose of the at least one vasodilator is nasally administered to the subject before the systemic administration of the dose of the at least one vasoconstrictor. In some embodiments, the dose of the at least one vasoconstrictor is systemically administered to the subject and the dose of the at least one vasodilator is nasally administered to the subject at approximately the same time (e.g., within 2 minutes or within 1 minute of each other).

In some embodiments, the dose of the at least one vasodilator is nasally administered and/or the dose of the at least one vasoconstrictor is systemically administered prior to the delivery of a chest compression to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of a chest compression to the subject). In some embodiments, the dose of the at least one vasodilator is nasally administered and/or the dose of the at least one vasoconstrictor is systemically administered after the initiation of chest compressions to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after the initiation of chest compressions to the subject). In some embodiments, the dose of the at least one vasoconstrictor is systemically administered to the subject and the dose of the at least one vasodilator is nasally administered to the subject at approximately the same time as the delivery of the chest compression (e.g., within 2 minutes or within 1 minute of each other).

In some embodiments, the dose of the at least one vasodilator is nasally administered and/or the dose of the at least one vasoconstrictor is systemically administered prior to the delivery of a defibrillating shock using a charged defibrillation device (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of a defibrillating shock to the subject). In some embodiments, the dose of the at least one vasodilator is nasally administered and/or the dose of the at least one vasoconstrictor is systemically administered after the delivery of a first defibrillating shock to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after delivery of a first defibrillating shock to the subject). In some embodiments, the dose of the at least one vasoconstrictor is systemically administered to the subject and the dose of the at least one vasodilator is nasally administered to the subject at approximately the same time as the delivery of the defibrillating shock (e.g., within 2 minutes or within 1 minute of each other).

In some embodiments, the dose of the at least one vasodilator is nasally administered and/or the dose of the at least one vasoconstrictor is systemically administered prior to the delivery of a defibrillating shock using a charged defibrillation device and prior to the performance of a chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of the later of the defibrillating shock and the chest compression to the subject). In some embodiments, the dose of the at least one vasodilator is nasally administered and/or the dose of the at least one vasoconstrictor is systemically administered after the delivery of a first defibrillating shock and the initiation of chest compressions to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after the later of the delivery of the first defibrillating shock or the initiation of chest compressions to the subject). In some embodiments, the dose of the at least one vasoconstrictor is systemically administered to the subject and the dose of the at least one vasodilator is nasally administered to the subject at approximately the same time as the delivery of the chest compression and the defibrillating shock (e.g., within 2 minutes or 1 minute of each other).

The dose of the at least one (e.g., at least two, three, four, five, or six) vasodilator can contain any of the exemplary vasodilators described herein or known in the art in any combination. The dose of the at least one vasodilator nasally administered to the subject can be any of the doses described herein or known in the art. The at least one vasodilator can be formulated for nasal administration using any of the techniques described herein or known in the art (e.g., an aerosol or a liquid formulation).

The dose of the at least one (e.g., at least two, three, four, five, or six) vasoconstrictors can contain any of the exemplary vasoconstrictors described herein or known in the art in any combination. The dose of the at least one vasoconstrictor systemically administered to the subject can be any of the doses described herein or known in the art. The at least one vasoconstrictor can be formulated for systemic administration using any of the techniques described herein or known in the art (e.g., a liquid formulation). In some embodiments, the systemic administration is intravenous, intaarterial, subcutaneous, intramuscular, subcutaneous, intraperitoneal, or intracardiac administration.

The methods described herein can include the systemic administration of one or more vasoconstrictors (e.g., any of the exemplary vasoconstrictors described herein and/or known in the art) and the nasal administration of one or more vasodilators (e.g., any of the exemplary vasodilators described herein and/or known in the art) in any combination without limitation.

Improvement in the efficacy of cardiac resuscitation can be determined by a health care professional by observing one or more physical parameters in the subject receiving cardiac resuscitation (a cardiac resuscitation protocol). Non-limiting examples of such parameters include: increased blood flow to one or more vital organs in the subject, increased blood pressure, increased blood flow to one or more of the subject's extremities, decrease in the length of time of cardiac resuscitation before heart contractions resume, a decrease in the number of defibrillating shocks administered to the subject prior to the restart of heart contractions, and a decrease in the amount of ischemia-reperfusion tissue damage in one or more tissues (e.g., one or more vital organs) in the subject. Additional examples of parameters for assessing the efficacy of cardiac resuscitation in a subject receiving cardiac resuscitation are known in the art. Methods for detecting these parameters are generally known by those in the art.

### Methods of Providing Cardiac Resuscitation

Also provided are methods for providing cardiac resuscitation to a subject having cardiac arrest that include: systemically administering a therapeutically effective dose of at least one (e.g., at least two, three, four, five, or six) vasoconstrictor to the subject; nasally administering a therapeutically effective dose of at least one (e.g., at least two, three, four, five, or six) vasodilator to the subject; and providing at least one (e.g., at least two, three, four, five, or six) defibrillating shock from a charged defibrillating device to the subject. Some embodiments further include providing at least one chest compression to the subject. In some embodiments, the dose of the at least one vasodilator is nasally administered to the subject within one hour (e.g., within 55, 50, 45, 40, 35, 30, 25, 20, 15, 10, 5, or 1 minute(s)) of the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) before the systemic administration of the vasoconstrictor. In some embodiments, the dose of the at least one vasodilator is administered to the subject no more than one hour (e.g., no more than 55, 50, 45, 40, 35, 30, 25, 20, 15, 10, 5, or 1 minute(s)) after the systemic administration of the vasoconstrictor.

Also provided are methods for performing cardiac resuscitation in a subject having cardiac arrest that include: systemically administering a therapeutically effective dose of at least one (e.g., at least two, three, four, five, or six) vasoconstrictor to the subject; nasally administering a therapeutically effective dose of at least one (e.g., at least two, three, four, five, or six) vasodilator to the subject; and providing at least one chest compression to the subject. In some embodiments, the dose of the at least one vasoconstrictor and the dose of the at least one vasodilator are administered within one hour (e.g., within 55, 50, 45, 40, 35, 30, 25, 20, 15, 10, 5, or 1 minute(s)) of each other.

In some embodiments, the dose of the at least one vasoconstrictor is systemically administered to the subject before the nasal administration of the dose of the at least one vasodilator. In some embodiments, the dose of the at least one vasodilator is nasally administered to the subject before the systemic administration of the dose of the at least one vasoconstrictor. In some embodiments, the dose of the at least one vasoconstrictor is systemically administered to the subject and the dose of the at least one vasodilator is nasally administered to the subject at approximately the same time (e.g., within 2 minutes or within 1 minute of each other).

In some embodiments, the dose of the at least one vasodilator is nasally administered and/or the dose of the at least one vasoconstrictor is systemically administered prior to the delivery of at least one chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of a chest compression to the subject). In some embodiments, the dose of the at least one vasodilator is nasally administered and/or the dose of the at least one vasoconstrictor is systemically administered after the initiation of chest compressions (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after the initiation of chest compressions to the subject). In some embodiments, the dose of the at least one vasoconstrictor is systemically administered to the subject and the dose of the at least one vasodilator is nasally administered to the subject at approximately the same time as the delivery of at least one chest compression or the initiation of chest compressions (e.g., within 2 minutes or within 1 minute of each other).

In some embodiments, the dose of the at least one vasodilator is nasally administered and/or the dose of the at least one vasoconstrictor is systemically administered prior to the delivery of at least one defibrillating shock using a charged defibrillation device (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of at least one defibrillating shock to the subject). In some embodiments, the dose of the at least one vasodilator is nasally administered and/or the dose of the at least one vasoconstrictor is systemically administered after the delivery of a first defibrillating shock to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after delivery of the first defibrillating shock to the subject). In some embodiments, the dose of the at least one vasoconstrictor is systemically administered to the subject and the dose of the at least one vasodilator is nasally administered to the subject at approximately the same time as the delivery of a defibrillating shock (e.g., a first defibrillating shock) (e.g., within 2 minutes or within 1 minute of each other).

In some embodiments, the dose of the at least one vasodilator is nasally administered and/or the dose of the at least one vasoconstrictor is systemically administered prior to the delivery of an defibrillating shock using a charged defibrillation device and prior to the performance of a chest compression (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute before delivery of the later of an defibrillating shock and a chest compression to the subject). In some embodiments, the dose of the at least one vasodilator is nasally administered and/or the dose of the at least one vasoconstrictor is systemically administered after the delivery of a first defibrillating shock and the initiation of chest compressions to the subject (e.g., no more than 1 hour, 55 minutes, 50 minutes, 45 minutes, 40 minutes, 35 minutes, 30 minutes, 25 minutes, 20 minutes, 15 minutes, 10 minutes, 5 minutes, or 1 minute after the later of the delivery of a first defibrillating shock or the initiation of chest compressions to the subject). In some embodiments, the dose of the at least one vasoconstrictor is systemically administered to the subject and the dose of the at least one vasodilator is nasally administered to the subject at approximately the same time as the delivery of the chest compression and the defibrillating shock (e.g., within 2 minutes or within 1 minute of each other).

The dose of the at least one (e.g., at least two, three, four, or five) vasodilator can contain any of the exemplary vasodilators described herein or known in the art in any combination. The dose of the at least one vasodilator nasally administered to the subject can be any of the doses described herein or known in the art. The at least one vasodilator can be formulated for nasal administration using any of the techniques described herein or known in the art (e.g., an aerosol or a liquid formulation).

The dose of the at least one (e.g., at least two, three, four, or five) vasoconstrictors can contain any of the exemplary vasoconstrictors described herein or known in the art in any combination. The dose of the at least one vasoconstrictor systemically administered to the subject can be any of the doses described herein or known in the art. The at least one vasoconstrictor can be formulated for systemic administration using any of the techniques described herein or known in the art (e.g., a liquid formulation). In some embodiments, the systemic administration is intravenous, intaarterial, subcutaneous, intramuscular, subcutaneous, intraperitoneal, or intracardiac administration.

The methods described herein can include the systemic administration of one or more vasoconstrictors (e.g., any of the exemplary vasoconstrictors described herein and/or known in the art) and the nasal administration of one or more vasodilators (e.g., any of the exemplary vasodilators described herein and/or known in the art) in any combination without limitation.

### Kits

Provided herein are kits include a composition containing at least one (e.g., at least two, three, four, five, or six) vasoconstrictor (e.g., any of the exemplary vasoconstrictors described herein and/or known in the art) that is formulated for systemic administration, and a composition containing at least one (e.g., at least two, three, four, five, or six) vasodilator (e.g., any of the exemplary vasodilators described herein or known in the art) that is formulated for systemic administration. In some embodiments, the composition containing at least one vasoconstrictor is formulated as a liquid for intravenous, intaarterial, subcutaneous, intramuscular, subcutaneous, intraperitoneal, or intracardiac administration.

Solutions or suspensions used for parenteral (e.g., intravenous, intaarterial, subcutaneous, intramuscular, subcutaneous, intraperitoneal, or intracardiac) administration can include the following components: a sterile diluent, such as water for injection, saline solution, fixed oils, polyethylene glycols, glycerine, propylene glycol, or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates, or phosphates; and agents for the adjustment of tonicity, such as sodium chloride or dextrose. pH can be adjusted with acids or bases, such as hydrochloric acid or sodium hydroxide. A parenteral preparation can be enclosed in ampules, disposable syringes, or multiple dose vials made of glass or plastic. In some embodiments, the at least one vasoconstrictor is provided in a pre-loaded syringe in a single dose.

Pharmaceutical compositions suitable for injectable use include sterile aqueous solutions (where water soluble) or dispersions and sterile powders for the extemporaneous preparation of sterile injectable solutions or dispersion. For systemic (e.g., intramuscular) administration, suitable carriers include physiological saline, bacteriostatic water, Cremophor EL™ (BASF, Parsippany, N.J.), or phosphate buffered saline (PBS). In all cases, the composition must be sterile and should be fluid to the extent that easy syringability exists. It should be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols, such as manitol, sorbitol, and sodium chloride in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation can include vacuum drying or freeze-drying which yields a powder of the active ingredient plus any additional desired ingredient from a previously sterile-filtered solution thereof.

In some embodiments, the composition containing at least one vasodilator is formulated as a liquid or as an aerosol for nasal administration. In some embodiments, the at least one vasodilator is formulated as an aerosol and is provided in a nebulizer. In some embodiments, the at least one vasodilator is provided in a therapeutically effective dose. For nasal administration, the at least one vasodilator can delivered in the form of an aerosol spray (e.g., a nasal spray) from pressured container or dispenser which contains a suitable propellant, e.g., a gas such as carbon dioxide, or a nebulizer. In some embodiments, the at least one vasodilator is formulated with one or more penetrants, including, for example, detergents, bile salts, and fusidic acid derivatives.

The composition containing at least one vasodilator and the composition containing at least one vasoconstrictor can each be formulated in a single dose form (e.g., any of the exemplary single dose concentrations described herein or known in the art for vasodilators and vasoconstrictors).

The kits can contain one or more vasoconstrictors (e.g., any of the exemplary vasoconstrictors described herein and/or known in the art) and one or more vasodilators (e.g., any of the exemplary vasodilators described herein and/or known in the art) in any combination without limitation.

In some embodiments, the kits provided herein further include instructions for performing one or more of the methods described herein (e.g., method of improving circulation in a subject undergoing a cardiac resuscitation protocol, methods of reducing the risk of stroke in a subject undergoing a cardiac resuscitation protocol, and/or methods of improving the efficacy of cardiac resuscitation in a subject undergoing a cardiac resuscitation protocol).

In some embodiments, the kits further include a defibrillating device or instructions for performing CPR.

A number of embodiments of the invention have been described. Nevertheless, it will be understood that various modifications may be made without departing from the spirit and scope of the invention. Accordingly, other embodiments are within the scope of the following claims.

1. A method for improving circulation in a subject undergoing a cardiac resuscitation protocol that includes systemic administration of a vasoconstrictor, the method comprising:
nasally administering a therapeutically effective dose of at least one vasodilator to a subject undergoing a cardiac resuscitation protocol that includes systemic administration of a vasoconstrictor,
wherein the nasal administration of the dose of the at least one vasodilator results in improved circulation in the subject as compared to control subject.

2-3. (canceled)

4. A method of improving circulation in a subject undergoing a cardiac resuscitation protocol, the method comprising:
systemically administering a therapeutically effective dose of at least one vasoconstrictor to a subject undergoing a resuscitation protocol; and
nasally administering a therapeutically effective dose of at least one vasodilator to the subject,
wherein the systemic administration of the dose of the at least one vasoconstrictor and the nasal administration of the dose of the at least one vasodilator improves circulation in the subject as compared to a control subject.

5-6. (canceled)

7. A method for providing cardiac resuscitation to a subject having cardiac arrest, the method comprising:
systemically administering a therapeutically effective dose of at least one vasoconstrictor to the subject;
nasally administering a therapeutically effective dose of at least one vasodilator to the subject;
providing at least one chest compression to the subject; and
providing at least one defibrillating shock from a charged defibrillating device to the subject.

8. (canceled)

9. The method of claim 7, wherein the at least one vasoconstrictor is selected from the group consisting of: tyramine, vasopressin, metaraminol, mephentermine, an amphetamine, methylphenidate, mephedrone, hydroxephedrine, angiotensin II, methoxamine, an antihistamine, oxymetazoline, propylhexedrine, caffeine, tetrahydrozoline hydrochloride, psilocybin, cocaine, lysergic acid diethylamide (LSD), lysergic acid amide (LSA), a muscarinic agonist, an adrenergic agonist, thromboxane, endothelin, asymmetric dimethylarginine, antidiuretic hormone (ADH), vasopressin, endotoxin, and thrombin.

10. The method of claim 7, wherein the at least one vasoconstrictor is administered intraperitoneally, intramuscularly, subcutaneously, intravenously, intraarterially, or intracardially.

11. The method of claim 7, wherein the at least one vasodilator is selected from the group consisting of: a calcium channel blocker, prostacyclin, hydralazine, minoxidil, quinapril, ramipril, a nitric oxide donor, sildenafil, tadalafil, vardenafil, tetrahydrocannabinol, theobromine, papaverine, estrogen, endothelium-derived hyperpolarizing factor (EDHF), nitric oxide, noradrenaline, histamine, prostacyclin, prostaglandin D₂, prostaglandin E₂, vasoactive intestinal peptide (VIP), L-arginine, bradykinin, substance P, niacin, platelet activating factor (PAF), a natriuretic peptide, prostaglandin I₂, prostaglandin E₂, heparin, fosinopril, lisinopril, enalapril, an alpha blocker, amyl nitrate, capsaicin, and ethanol.

12. The method of claim 7, wherein the dose of the at least one vasodilator is formulated as a liquid.

13. The method of claim 7, wherein the dose of the at least one vasodilator is formulated as an aerosol.

14. The method of claim 1, wherein the cardiac resuscitation protocol that includes systemic administration of a vasoconstrictor includes at least one defibrillating shock from a charged defibrillating device.

15. The method of claim 1, wherein the cardiac resuscitation protocol that includes systemic administration of a vasoconstrictor includes at least one chest compression.

16-17. (canceled)

18. The method of claim 7, wherein the dose of the at least one vasodilator is administered to the subject within 5 minutes of the systemic administration of the vasoconstrictor.

19-21. (canceled)

22. The method of claim 7, wherein the dose of the at least one vasoconstrictor and the dose of the at least one vasodilator are administered within 10 minutes of each other.

23. The method of claim 7, wherein the dose of the at least one vasoconstrictor and the dose of the at least one vasodilator are administered within 5 minutes of each other.

24. (canceled)

25. The method of claim 7, wherein the at least one defibrillating shock is provided to the subject within 10 minutes of administering the dose of the at least one vasoconstrictor and/or within 10 minutes of administering the dose of the at least one vasodilator.

26. The method of claim 7, wherein the at least one defibrillating shock is provided to the subject within 5 minutes of administering the dose of the at least one vasoconstrictor and/or within 5 minutes of administering the dose of the at least one vasodilator.

27-28. (canceled)

29. The method of claim 4, further comprising providing at least one chest compression to the subject.

30-34. (canceled)

35. A kit comprising:
a composition comprising at least one vasoconstrictor that is formulated for systemic administration; and
a composition comprising at least one vasodilator that is formulated for nasal administration.

36. The kit of claim 35, wherein the composition comprising at least one vasodilator is formulated as a liquid.

37. The kit of claim 35, wherein the composition containing at least one vasodilator is formulated as an aerosol.

38. The kit of claim 35, further comprising a nebulizer that contains the aerosol.

39. The kit of claim 35, wherein the composition containing at least one vasoconstrictor is formulated in a therapeutically effective dose.

40. The kit of claim 35, wherein the composition containing at least one vasodilator is formulated in a therapeutically effective dose.

41. The kit of claim 35, further comprising a defibrillating device.
